(19) United States
(12) Patent Application Publication
(10) Pub. No.: US 2002/0045723 A1
(43) Pub. Date: Apr. 18, 2002

(54) PROCESS FOR THE PRODUCTION OF ETHER ISOCYANATES BACKGROUND OF THE INVENTION

(76) Inventors: FRANK RICHTER, LEVERKUSEN (DE); JOSEF PEDAIN, KOLN (DE); KLAUS KACHTKAMP, DUSSELDORF (DE); ANDREAS FLINK, DORMAGEN (DE)

Correspondence Address: PATENT DEPARTMENT, BAYER CORPORATION, 100 BAYER ROAD, PITTSBURGH, PA, 152059741

(21) Appl. No.: 08/713,905

(22) Filed: Sep. 13, 1996

(30) Foreign Application Priority Data
Sep. 25, 1995 (DE) 19535506.7

(51) Int. Cl. C08G 18/77; C07C 265/00

(52) U.S. Cl. 528/44; 560/330; 568/583

(57) ABSTRACT

Ether isocyanates are prepared by phosgenation of ether amines in the vapor phase.

### BACKGROUND OF THE INVENTIQN

The present invention relates to a process for the production of ether (poly)isocyanates by phosgenation of ether amines in the vapor phase.

Isocyanates containing ether groups or ether isocyanates are well known. (See, for example, Annalen der Chemie, 562 (1949), 83 ff). However, these known ether-containing isocyanates are generally obtainable only in poor yield and low purity.

In the reactions of ether amines, chlorinated products are frequently obtained by splitting the ether (U.S. Pat. No. 3,267,122). Thus, for example, H₂N(CH₂)₃—O—(CH₂)₄—O—(CH₂)₃NH₂ is split into OCN(CH₂)₃Cl during the reaction with COCl₂ (reference: Angew. Chem., A 59 (1949), 271).

Only aliphatic ether amines having HCl salts which are soluble in chlorinated hydrocarbons, for example, C₄H₉—O—(CH₂)₃NH₂, can be converted to ether isocyanates at temperatures below 80° C. (Annalen der Chemie, 562 (1949), 105). The yield of this isocyanate is, however, only 86% of the theoretical yield. In such a process, it is necessary to convert the amine into the amine hydrochloride prior to the phosgenation. About 80% methoxypropylamine hydrochloride splits at 140 to 150° C. in 1-chloronaphthalene with phosgene to form 3-chloropropyl isocyanate (Annalen der Chemie, 562 (1949), 104).

Certain ether isocyanates can be obtained in yields of up to about 80% by simple base phosgenation. (See, for example, DE-A 1,154,092.) However, the products of such processes have very high residual chlorine contents (0.1%). Such a high chlorine content in the diisocyanate frequently makes it difficult to use those products. For example, such chlorine-containing diisocyanates are not useful for the preparation of non-discoloring raw materials for coatings. The corresponding hydrochlorides of the amines or carbamates must be used in such processes. The handling of heterogeneous reaction mixtures of this type is, however, very difficult and is an obstacle to the smooth, economical production of the isocyanates.

(Poly)isocyanates containing ether groups are also obtainable by nucleophilic substitution of organic halides by metal cyanates. (See, for example, JP 50 036 424; Arch. d. Pharm., 302 (1969), 617; and DE-A 2,031,291) The accumulation of salts, the generally low conversion rates and the environmental problems encountered in these processes are obstacles to the industrial exploitation of this approach.

The reaction of ether (poly)amines with low-molecular weight alkyl isocyanates, subsequent thermal decomposition of the ureas formed and separation of alkylamine has also been proposed as a method for the preparation of ether (poly)isocyanates (“isocyanate interchange”; see, for example, DE-A 3,232,917). However, this method has several disadvantages. First, a by-product which must be disposed of is obtained. Second, considerable quantities of urea remain in the product, particularly when the ether (poly)isocyanates cannot be worked up by subsequent purification processes. Another disadvantage of this process is that the “isocyanate interchange” is a typical equilibrium reaction and is therefore difficult to carry out quantitatively.

Isocyanates containing ether groups can also be prepared by Curtius rearrangement of the corresponding carboxylic acid azides (J. Prakt. Chem., 335 (1993), 294 and the references cited therein), but only on a laboratory scale.

As is explained in DE-A 1,165,580, e.g., polyisocyanates containing ether groups are of great interest for use in paints and coatings.

Splitting the ether group in ether amines occurs more readily and completely at elevated temperatures. For example, phosgenation of 3-methoxypropylamine in toluene below 110° C. produces a mixture of 3-methoxypropyl isocyanate and 3-chloropropyl isocyanate. At elevated temperature (e.g., 140 to 150° C.) in chloronaphthalene as solvent, however, substantially only 3-chloropropyl isocyanate is formed (Annalen der Chemie, 562 (1949), 83).

DE-A 1,793,329 discloses a cold phase-hot phase phosgenation in solution for the preparation of ether(poly)isocyanates. It is alleged that very little, if any, splitting of the ether occurs. However, the yields of isocyanate are only 60 to 75% of the theoretical yield. The chlorine content of the products, at 400 to 2000 ppm, is far too high for many applications, particularly for paint and coatings applications.

### SUMMARY OF THE INVENTION

It is an object of the present invention to provide a process for the production of ether (poly)isocyanates.

It is also an object of the present invention to provide a simple process for the production of isocyanates containing ether groups.

It is another object of the present invention to provide a process for the production of high quality isocyanates containing ether groups.

It is a further object of the present invention to provide a process for the production of high quality ether isocyanates in high yields without significant product loss through, for example, splitting of the ether groups

These and other objects which will be apparent to those skilled in the art are accomplished by converting mono- and polyamines containing ether groups to the corresponding isocyanates in very good yields and in high purity, without splitting the ether group. This conversion is achieved by reacting an ether-containing amine in the vapor phase with phosgene in the vapor phase under applied pressure at a temperature in the range from 50 to 800° C., preferably from 100 to 550° C. (depending on the boiling point of the amine), optionally in the presence of an inert carrier gas.

### DETAILED DESCRIPTION OF THE INVENTION

The present invention relates to a method for preparing ether (poly)isocyanates from ether (poly)amines in which ether (poly)amines are reacted with at least the stoichiometric quantity, based on the NH₂ group(s), of phosgene or corresponding quantities of a material which generates phosgene, in the vapor phase close to or above the boiling point of the starting (poly)amine which boiling point is in the temperature range of from 50 to 800° C., preferably from 100 to 550° C., under applied pressure.

In the present invention, the ether amines which may be used include compounds represented by Formula (I)

X(—R^{1—O—R}²—NH₂)ₙ (I),

in which

x represents H, NH₂ or C(R³)₄₋ₙ,

R¹, R² and R³ each represent the same or a different, optionally branched, optionally substituted (e.g., with Cl, Br), optionally heteroatom-containing (e.g., N, 0, S)C₁-C₁₀alkyl, C₃-C₂₄ cycloalkyl, C₇-C₂₄ aralkyl, or C₆-C₂₄ aryl radical, and R¹ may also represent a direct bond between X and the ether oxygen atom bonded to R², and

n represents 1, 2 or 3.

The process of the present invention may be carried out using known techniques. Suitable techniques are disclosed in EP-A 0,570,799 and DE-A 4,412,327. In these disclosed processes, the co-reactants are introduced into suitable reactors maintained at a temperature close to or above the boiling point of the starting amine or mixture of amines. The co-reactants are then mixed and reacted with one another. The temperature, depending upon the pressure, is generally between 50 and 800° C., preferably between 100 and 550° C. The process is generally carried out within a pressure range of from 10 mbar to 5 bar, preferably from 200 mbar to 3 bar.

Introduction of the reaction components during the vapor phase phosgenation may optionally take place in the presence of inert additives such as carrier gases. The carrier gases used may be nitrogen, argon or other inert gases and vapors of commercially available solvents such as chlorobenzene, dichlorobenzenes, xylenes, chloronaphthalenes and decahydronaphthalene.

The phosgene used in the phosgenation reaction is used in a stoichiometric amount or in stoichiometric excess, determined on the basis of the number of primary amino groups in the amine starting material. A quantity of phosgene amounting to from 100 to 300% of the theoretical quantity, preferably from 100 to 200% of the theoretical quantity, is generally sufficient.

After the reaction with phosgene, the ether isocyanates are recovered by cooling the gas stream to a temperature above the decomposition temperature of the corresponding intermediate carbamic acid chlorides. The ether isocyanate may then be isolated in pure form by known processes such as distillation, crystallization, extraction or film distillation, or recovered as raw product (solution).

The amine starting materials which are converted into the corresponding isocyanates by the process of the present invention may be obtained by a number of known processes. One suitable known process is alkoxylation of water or of other, optionally polyfunctional, OH-functional compounds such as alcohols, phenols and/or carboxylic acids and subsequent amination (for example, FR-A 1 361 810). Another suitable process for producing the amine starting material is polymerization of tetrahydrofuran and, optionally after further reaction with alkylene oxide, subsequent treatment as described in FR-A-1 361 810. Suitable amine starting materials may also be produced by cyanoethylation of water and subsequent hydrogenation to form bis(3-aminopropyl)ether (DRP 731 708) or by cyanoethylation of other, optionally polyfunctional, OH-functional compounds (particularly diols and triols) and subsequent hydrogenation.

The usefulness of mono- and polyamines containing ether groups in the phosgenation of the present invention is determined essentially by the vapor pressure of the amine at the applied pressure. In the case of particularly high-boiling compounds, it may be advantageous to introduce the amine into the phosgenation reaction as an azeotrope with other substances, or to use a carrier gas for the introduction of the amine component into the reaction chamber.

Typical examples of suitable (poly)amines of Formula (I) which may be used alone or as mixtures include: alkyl aminoalkyl ethers such as aminomethyl methyl ether, aminomethyl ethyl ether, aminomethyl propyl ether (as well as isomers), 1-aminoethyl methyl ether, 2-amino ethyl methyl ether, and aminopropyl methyl ether (as well as isomers); diamino-oxoalkanes such as 1,1′-bis(aminomethyl) ether, 1,1′-bis(amino ethyl)ether, 1,2′-bis(amino-ethyl) ether, 2,2′-bis(amino-ethyl) ether and technical mixtures of the three latter diamines, bis(aminopropyl) ether (all isomers, optionally as a mixture), diamino(poly)oxoalkanes such as 1,8-diamino-1,5,8-trimethyl-3,6-dioxaoctane, 1,11-diamino-1,5,8,11-tetramethylundecane and all isomers of the two latter compounds having vicinal O—N bonding in pure form or as a mixture (for example, as commercial Jeffamine D 230), 1,8-diamino-3,6-dioxaoctane (for example, as commercial Jeffamine EDR 148), 1,10-diamino-4,7-dioxadecane, 1,12-diamino-4,9-dioxadodecane, 1,14-diamino-3,10-dioxatetradecane, and 1,13-diamino-4,7,10-trioxatridecane; triamino(poly)oxoalkanes such as 1,7-diamino-2,6-dioxa-4-aminomethoxyheptane, 1-amino-2-oxa-3,3-bis(aminomethoxy)hexane, 1,9-diamino-3,7-dioxa-5-(1-amino-2-ethoxy)-nonane, 1-amino-3-oxa-4,4′-bis(1-amino-2-ethoxy)heptane, 1,11-diamino- 4,8-dioxa-6-(1-amino-5-oxobutyl)undecane, 1-amino-4-oxa-5,5-bis(1-amino-5-oxobutyl)octane and mixtures of the above-mentioned mono-amines, diamines and triamines.

The mixture of isomers composed of 2-(2-isocyanatopropoxy)-1-propyl isocyanate, 1,1′-oxydi-2-propyl isocyanate and 2,2′-oxydi-1-propyl isocyanate (“dipropylene glycol diisocyanate”, mixture of isomers) prepared by the process of the present invention is new.

The ether isocyanates prepared by the process of the present invention are valuable raw materials for the production of polyurethanes (optionally foamed), adhesives, coating materials, emulsifiers, thickeners, oligomeric isocyanate modification products (e.g., polyisocyanates containing uretdione, isocyanurate, carbodiimide, biuret, urethane and allophanate groups), and auxiliary substances which are used, for example, for imparting wet strength to paper and other cellulose products. These ether isocyanates are useful as raw materials for the production and/or formulation of active substances and pharmaceuticals (DE-A 3,232,917).

The invention is further illustrated but is not intended to be limited by the following examples in which all parts and percentages are by weight, unless otherwise specified.

### EXAMPLES

### Example 1

2-(2-isocyanatopropoxy)-1-propyl isocyanate, 1,1′-oxydi-2-propyl isocyanate and 2,2′-oxydi-1-propyl isocyanate (“dipropylene glycol diisocyanate”, mixture of isomers) were prepared by the procedure described below.

The apparatus in which the reaction was conducted included a mixer tube heated to 400° C. which was 2.5 mm in diameter and 17.5 mm in length having a condensation stage arranged in tandem and a connected COCl₂ adsorption tower filled with activated carbon. COCl₂, which had been heated to 420° C. at 950 mbar in a heat exchanger connected in front, flowed continuously at a rate of 2.5 mol/h through a nozzle projecting into the mixer tube. Simultaneously, a mixture of amines heated to 320° C., obtained by catalytic amination under pressure of technical dipropylene glycol (approx. 50% 2-(2-hydroxypropoxy)-1-propanol, approx. 40% 1,1′-oxydi-2-propanol and approx. 10% 2,2′-oxydi-1-propanol) having a boiling range of 72 to 78° C. at a pressure of 7.5 mbar, was introduced at a feed rate of 1 mol/h, together with dry nitrogen at a rate of 0.1 mol/h as diluent, into the reaction chamber via the annular passage between the nozzle and the mixer tube. A pressure of approx. 350 mbar was maintained in the mixer tube by applying a reduced pressure at the end of the condensation stage. That is, the reaction mixture leaving the reaction chamber was passed in a condensation stage through 1,2-dichlorobenzene, which was maintained at 150 to 160° C. Here the selective condensation of the diisocyanates formed took place. In the adsorption tower, COCl₂ was separated from the gas mixture passing through the scrubbing stage and containing nitrogen, HCI and excess COCl₂. The mixture of diisocyanates was recovered in a pure state by distillation (Kp=95° C./0.05 mbar, n_{D}=1.4393/20° C.) and descended as a colorless liquid having an NCO content, titrated in accordance with DIN 53 185, of 45.4% (theoretical:46.6%). The yield of the pure, distilled mixture of diisocyanates was 98.2% of the theoretical yield, based on the mixture of diamines used, with a purity of 99.7% as determined by gas chromatography and a content of hydrolyzable chlorine of 43 ppm.

### Example 2

1,8-diisocyanato-3,6-dioxaoctane was prepared in accordance with the procedure described below.

2.5 kg (16.87 mol) of 1,8-diamino-3,6-dioxaoctane which is commercially available from Aldrich (also known as Jeffamine EDR 48) was converted into 1,8-diisocyanato-3,6-dioxaoctane and isolated in the manner specified in Example 1.

Yield: 3360 g=99.5% of the theoretical yield, purity (GC): 99.8% NCO content in accordance with DIN 53 185: 42.0% (theoretical: 42.0%), Hydrolyzable chlorine content: 48 ppm. Kp: 95° C./0.5 mbar.

### Example 3

1,12-diisocyanato-4,9-dioxadodecane was prepared from 2.5 kg (12.24 mol) of 1,12-diamino-4,9-dioxadodecane was prepared from 2.5 kg from Aldrich) and isolated in the manner specified in Example 1.

Yield: 3056 g=97.4% of the theoretical yield, purity (GC): 99.5% NCO content in accordance with DlN 53 185: 32.9% ) theoretical: 33.0%), Hydrolyzable chlorine content: 34 ppm. Kp: 83° C./0.2 mbar.

### Example 4

1,3-bis(3-isocyanatopropoxy)-2,2-dimethyl propane was prepared from 2.5 kg (11.45 mol) of 1,3-bis(3-aminopropoxy)-2,2-dimethyl propane (commercially available from Aldrich) and isolated in the manner specified in Example 1.

Yield: 3067 g=99.1% of the theoretical yield, purity (GC): 99.8% NCO content in accordance with DlN 53 185: 31.0% (theoretical: 31.1%), Hydrolyzable chlorine content: 24 ppm. Kp: 108° C./0.1 mbar.

### Example 5

3-methoxypropyl isocyanate was prepared from 1,000 g (11.2 mol) of 3-methoxypropylamine (commercially available form Aldrich) was converted into the isocyanate and isolated in the manner specified in Example 1.

Yield: 1250 g=96.8% of the theoretical yield, purity (GC): 99.1% NCO content in accordance with DlN 53 185: 36.5% (theoretical: 36.50%), Hydrolyzable chlorine content: 44 ppm. Kp: 55° C./20 mbar.

The identity of all the compounds produced in these Examples was deduced from lR, ¹H-NMR, ¹³C-NMR and mass spectroscopic analysis and from the results of elemental analysis.

### Comparative Examples Liquid Phosgenation

440 g of monochlorobenzene were mixed at 5° C. with 330 g of phosgene in a four-necked mixing flask equipped with reflux condenser, internal thermometer, dropping funnel and inlet tube. Then a solution of 71.5 g of the mixture of diamines specified in Example 1 in 900 g of monochlorobenzene was added dropwise over a period of 90 min.

The reaction mixture was then slowly heated, with stirring, to an internal temperature of 90° C. with simultaneous introduction of phosgene (approx. 1 mol/h) and maintained at this temperature for serveral hours. It was not possible to achieve a complete elucidation of the reaction mixture. After blowing off of the excess phosgene with nitrogen, filtration and working up be distillation, 19.5 g (19.6% of the theoretical yield) of a slightly colored liquid was obtained, having a boiling range of 80 to 85° C./0.07 mbar and an NCO content in accordance with DIN 53 185 of 42.2%.

Neither varying the solvent (1,2-dichlorobenzene (34% theoretical yield) and toluene (22.3% theoretical yield) nor converting the mixture of diamines into the dihydrochloride and bis(carbamate) (15.3% and 27.6% yields, respectively) increased the yield of diisocyanate substantially. The residual chlorine content of the product was in no case below 0.1%.

Further examples of phosgenations of ether diamines in the liquid phase are described, for example, in Annalen der Chemie, 562 (1949), 6 ff; DE-A 1,154,092; JP 4,027,365; FR 1,578,622.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the production of an ether (poly)isocyanate from an ether (poly)amine comprising reacting
a) an ether (poly)amine
with at least a stoichiometric amount (based o the number of primary amine groups present in a)) of
b) phosgene or a compound which generates phosgene under the reaction conditions
in the vapor phase at a temperature of fro about 50 to about 800° C. which temperature is close to or above the boiling point of a) under applied pressure.

2. The process of claim 1 in which ether (poly)amine a) is represented by the formula
X-(-R¹—O—R²—NH₂)n (I)
in which
X represents H, NH₂ or C(R³)₄₋ₙ,
R¹, R² and R³ each represents an optionally branched, an optionally substituted, or an optionally heteroatom-containing C₁-C₁₀, alkyl, C₃-C₂₄ cycloalkyl, C₇-C₂₄ aralkyl, or a C₆-C₂₄ aryl radical, provided that R₁ may also represent a direct bond of X to the ether oxygen atom bond d to R²,
and
n represents 1, 2 or 3.

3. The ether (poly)isocyanates selected from 2-(2)isocyanato-propoxy)-1-propyl isocyanate, 1,1′-oxydi-2-propyl isocyanate, 2,2′-oxydi-1-propyl isocyanate and mixtures thereof.

4. A process for the production of a polyurethane comprising reacting the ether (poly)isocynate produced in accordance with claim 1 with an isocyanate-reactive material. l each represents and optionally branched, an
